(19) United States
(12) Patent Application Publication
(10) Pub. No.: US 2025/0059331 A1
(43) Pub. Date: Feb. 20, 2025

(54) METHOD FOR PRODUCING OXYPROPYLENE GROUP-CONTAINING GLYCOL ETHER HAVING REDUCED CONTENT OF ALLYL GROUP-CONTAINING IMPURITIES

(71) Applicant: DOW TORAY CO., LTD., Shinagawa-Ku, Tokyo (JP)

(72) Inventors: Son Thanh PHAN, Ichihara-shi, Chiba (JP); Seiki TAMURA, Ichihara-shi, Chiba (JP); Hiroyuki INAGAKI, Ichihara-shi, Chiba (JP)

(21) Appl. No.: 18/722,516

(22) PCT Filed: Dec. 15, 2022
(86) PCT No.: PCT/JP2022/046182

§ 371 Date: Jun. 20, 2024

(30) Foreign Application Priority Data
Dec. 21, 2021 (JP) 2021-206680

(51) Int. Cl. C08G 77/46; C07C 41/46; C08G 18/82

(52) U.S. Cl. CPC C08G 77/46; C07C 41/46; C08G 18/82

(57) ABSTRACT

Provided is a method to reduce allyl group-containing impurities in an oxypropylene group-containing glycol ether using a relatively easy and simple industrial facility and reaction process, to enable the hydrosilylation reaction products and oxypropylene group-containing glycol ether to be easily separated by distillation, or the like, to prevent the oxypropylene group-containing glycol ether from adversely affecting the polymerization reaction when used as a reaction solvent or diluent for polyether-polysiloxane block copolymers, and to easily produce the oxypropylene group-containing glycol ether at low cost. A method for producing oxypropylene group-containing glycol ethers, comprises a step of inducing a hydrosilylation reaction of an allyl group-containing impurity included in an oxypropylene group-containing glycol ether and a silicon atom-bonded hydrogen atom (Si—H)-containing compound. Optionally, an additional purification step is utilized for separating the oxypropylene group-containing glycol ether by distillation or the like.

### TECHNICAL FIELD

The present invention relates to a method for producing an oxypropylene group-containing glycol ether with reduced allyl group-containing impurities, and in particular, to a method for producing the glycol ether using an additional purification step, or the like.

### BACKGROUND ART

A method of obtaining a polyoxyalkylene derivative by reacting starting materials containing a hydroxyl group with an alkylene oxide using an alkali metal hydroxide such as potassium hydroxide as a catalyst is industrially important and has been known for many years. Patent Document 1 reported that when polyether polyol is produced by addition polymerization of propylene oxide to a polyhydric alcohol, a small amount of unsaturated groups are detected in the product. It is not possible to remove the impurity by a normal distillation or stripping process due to the high molecular weight of the impurity.

Patent Document 2 describes the reaction mechanism of the byproduction of unsaturated compounds during ring-opening addition polymerization of propylene oxide in the presence of a strong alkali catalyst. In other words, “Using a customary catalyst such as KOH, for example, a small amount of PO is continuously converted to form allyl alcohol, which serves as a new source of unsaturated initiator that competes with the original initiator (starter). Finally, conditions are established such that the addition of additional PO cannot increase the overall molecular weight of the polyether product”.

Glycol ether has long been produced using a similar reaction mode, but since these are low molecular weight compounds, they are usually separated through a distillation process and supplied to the market as a high-purity raw material due to differences in boiling point determined by the degree of polymerization (1, 2, or 3). Patent Document 3 discloses a process characterized by use of a nonprotic polar solvent that can improve the conversion rate.

On the other hand, it has long been known that glycol ether is susceptible to quality deterioration due to oxidation, and indicators thereof include odor, peroxide value, carbonyl value, acid value, increased UV absorbance, and the like. Patent Document 4 discloses that treatment with a metal hydride such as NaBH₄, KBH₄, and LiAlH₄ and the like (decomposition treatment of oxidation-degradation products by reduction reaction) is superior to activated carbon treatments and water distillation in terms of improving these properties. However, metal hydrides are water-prohibited substances and are extremely reactive, making them difficult to handle and difficult to apply to industrial-scale production.

Patent Document 5 reported that the amount of aldehydes, peroxide value, and UV absorbance can be reduced by the action of hydrogen gas on glycol ether in the presence of a hydrogenation catalyst. The nickel-diatomite powder catalyst used here is generally stable in air and has the advantage of being easier to apply in industrial production, as compared to the metal hydride of Patent Document 4. However, the hydrogenation reaction requires special equipment because of the use of highly combustible/flammable hydrogen gas, resulting in a significant increase in processing costs and the consequent difficulty in market dissemination and production capacity limitations.

Patent Document 6 discloses a method for purifying propylene glycol monoalkyl ether produced by the reaction of propylene oxide with alcohol in the presence of an alkali or alkaline earth metal alkoxide by activated carbon treatment. Thereby a reduction in carbonyl impurities and UV absorbance can be achieved.

Patent Document 7 discloses a specific manufacturing process for processing propylene glycol monoalkyl ether obtained by the reaction of propylene oxide with alcohol in the presence of an alkali or alkaline earth metal alkoxide using an alkali metal borohydride (for example, NaBH₄). Thereby a reduction in carbonyl impurities and UV absorbance can be achieved.

Patent Document 8 discloses a foam inhibitor composition containing: (A) a specific silicone oil compound; (B) a mixture of a “polyhydric alcohol alkyl ether with a molecular weight of 50 to 500” such as dipropylene glycol monomethyl ether or the like and a specific side chain polyoxyalkylene modified silicone oil; and (C) a surfactant other than the polyoxyalkylene-modified silicone oil, in a specific ratio. Furthermore, an example of synthesis of the polyoxyalkylene-modified silicone oil in the presence of dipropylene glycol monomethyl ether is described.

Patent Document 9 discloses a composition containing a specific side-chain polyether-modified silicone and a specific glycol ether compound, a manufacturing method thereof, and a foam conditioner for polyurethane foam containing the composition. Furthermore, an example of the use of dipropylene glycol monobutyl ether as a reaction solvent and diluent for the production of the aforementioned polyether-modified silicone is disclosed.

Patent Document 10 discloses a composition containing (A) a nonhydrolyzable (AB)n type polyether-modified silicone and (B) a specific glycol ether compound in which terminal hydrogen is substituted with a hydrocarbon group having 1 to 8 carbon atoms and which has a secondary alcoholic hydroxyl group at the other terminal, a method for manufacturing the composition, a foam stabilizer for polyurethane foam containing the composition, and the like. Furthermore, an example is disclosed of the use of the aforementioned component (B) as a reaction solvent and diluent for the production of component (A). However, these documents do not describe nor suggest any of the new technical problems discussed below.

As described above, oxypropylene group-containing glycol ethers have conventionally been distilled and refined, and supplied to the market as high-purity products. However, in these documents, there is no mention nor suggestion that the oxypropylene group-containing glycol ether contains allyl group containing-impurities and that these impurities are extremely difficult to remove by ordinary distillation and purification methods, nor of any technical disadvantages derived from these impurities. Therefore, the technical problem of the invention was not recognized.

Note that Patent Document 5 discloses a method of converting an allyl group to an inert propyl group, but using this method in an industrial large-scale production process would lead directly to a significant increase in production costs and a reduction in production capacity. Therefore, an industrially cheaper and simpler method is required to reduce allyl group containing-impurities.

### RELATED ART DOCUMENTS

### Patent Documents

Patent Document 1: Japanese Examined Patent Application No. S42-13762 (U.S. Pat. No. 3,370,056)
Patent Document 2: U.S. Pat. No. 5,856,369 (U.S. Pat. No. 3,971,716)
Patent Document 3: International Patent Application WO 1996/036582
Patent Document 4: German Patent No. 2831210 Specification
Patent Document 5: Japanese Patent No. 2553146
Patent Document 6: U.S. Pat. No. 7,439,405 (U.S. Pat. No. 5,859,730)
Patent Document 7: U.S. Pat. No. 9,187,392 (U.S. Pat. No. 6,460,434)
Patent Document 8: Japanese Patent No. 3570490 (U.S. Pat. No. 6,417,258)
Patent Document 9: U.S. Pat. No. 11,066,534 Specification (WO2018074257A1)
Patent Document 10: U.S. Pat. No. 10,717,872 (U.S. Pat. No. 6,655,066)

### SUMMARY OF THE INVENTION

### Problem to be Solved by the Invention

On the other hand, when the present inventors examined the use of low-cost glycol ether compound raw materials in order to provide the foam conditioner of patent document 10 at a lower cost, they found an unreported and new technical problem that the molecular weight of component (A) did not reach the target, and the viscosity of the foam conditioner was very low due to problems arising from the quality of the glycol ether compound (especially, the presence of allyl group containing-impurities that had not been recognized).

More specifically, compositions containing (AB)n-type polyether-modified silicone and specific glycol ether compounds, foam regulators, and the like, as previously proposed by the present inventors in Patent Document 10, are expected to exhibit excellent effects as surfactants for foam control or foam stabilization in various polyurethane foam formulations, and can be manufactured by a simple process, which is advantageous for industrial production and mass supply. However, the present inventors have discovered a new problem for obtaining this composition, namely, the quality of the glycol ether compound has a significant impact on the quality during production. In other words, low-cost glycol ether compounds contain a large amount of allyl group containing-impurities, which cause side reactions that seal the growing end of the copolymer during (AB)n-type polyether modified silicone synthesis, resulting in a low viscosity composition after hydrosilylation is completed.

The particularly important role of (AB)n-type polyether-modified silicone foam stabilizer is for foam retention during polyurethane foam formation, and a conventional method for improving foam retention designs a foam stabilizer formulation so as to obtain a high-molecular-weight and high-viscosity product having a large n number, or in other words, to react the starting polyether and polysiloxane with a C═C/SiH molar ratio of approximately 1.0. However, if the glycol ether compound, which is the reaction solvent, contains more than a certain amount of allyl group-containing impurities, achieving the target molecular weight of the copolymer will be difficult. In addition, the addition of allyl group-containing impurities as a variable factor in the reaction system increases the complexity of product design and quality control for foaming agents.

Due to this complex and complicated issue, (AB)n polyether-modified silicone foaming agents still have problems such as insufficient market penetration despite their potential value. Therefore, development of a method for reducing allyl group-containing impurities, especially in oxypropylene group-containing glycol ethers, and a method for producing purified glycol ethers with reduced allyl group-containing impurities, are needed for a stable supply of the foaming agents to the market.

### Means for Solving the Problem

The present inventors achieved the present invention by discovering that the aforementioned problems can be resolved by a method for producing an oxypropylene group-containing glycol ether which involves a step of inducing a hydrosilylation reaction of an allyl group containing-impurity included in the aforementioned oxypropylene group-containing glycol ether and a silicon atom-bonded hydrogen atom (Si—H)-containing compound. After the hydrosilylation reaction step, the purity of the oxypropylene group-containing glycol ether is 90 mass % or more, more preferably 95 mass % or more, and is substantially free of allyl group-containing impurities. Furthermore, a purification process to separate the oxypropylene group-containing glycol ether from the aforementioned hydrosilylation reaction product by means of distillation or the like is particularly preferably added after the aforementioned hydrosilylation reaction step.

Furthermore, the inventors also found that the aforementioned problems were solved by a method of producing a polyether-polysiloxane block copolymer composition or a polyurethane foam-forming composition, wherein the oxypropylene group-containing glycol ether obtained by the aforementioned production method is used as a reaction solvent or diluent, and thus achieved the present invention.

### Effect of the Invention

The present invention can reduce allyl group-containing impurities in an oxypropylene group-containing glycol ether using a relatively easy and simple industrial facility and reaction process, enable the hydrosilylation reaction products and oxypropylene group-containing glycol ether to be easily separated by distillation, or the like, prevent the oxypropylene group-containing glycol ether from adversely affecting the polymerization reaction when used as a reaction solvent or diluent for high purity polyether-polysiloxane block copolymers having an (AB)n type polyether modified silicone, and can easily produce the oxypropylene group-containing glycol ether at low cost. Thereby, even relatively inexpensive, low-quality oxypropylene group-containing glycol ether, which has a large amount of impurities, can use an industrially low-cost and easy purification method, and use of these purified raw materials can mitigate the effect on the quality of these raw materials and facilitate product design, quality control, and stable and inexpensive supply of surfactants and the like (especially foaming agents) that primarily contain high-performance (AB)n-type polyether modified silicone, and enable the widespread use of these products in the market.

### DESCRIPTION OF THE PREFERRED EMBODIMENTS

The following is a more detailed description of the method for producing oxypropylene group-containing glycol ether of the present invention.

In the present invention, “oxypropylene group-containing glycol ether” refers to glycol ethers containing oxypropylene groups in which a terminal hydrogen is substituted by a hydrocarbon group having 1 to 8 carbon atoms and an alcoholic hydroxyl group is provided at the other end, the number of repeating oxyalkylene units having 2 to 4 carbon atoms is in a range of 1 to 3 and no heteroatoms other than oxygen are included. These components are useful as reaction solvents or diluents for polyether-polysiloxane block copolymers, including (AB)n-type polyether modified silicone, as described in Patent Document 10.

In the present invention, the oxypropylene group-containing glycol ether can be one or more type of glycol ether selected from propylene glycol monobutyl ethers, dipropylene glycol monobutyl ethers, tripropylene glycol monobutyl ethers, propylene glycol monomethyl ethers, dipropylene glycol monomethyl ethers, tripropylene glycol monomethyl ethers, propylene glycol monopropyl ethers, dipropylene glycol monopropyl ethers, tripropylene glycol monopropyl ethers, propylene glycol monoethyl ethers, dipropylene glycol monoethyl ethers, and tripropylene glycol monoethyl ethers. In particular, the oxypropylene group-containing glycol ether of the present invention is preferably 1 or more type of glycol ether selected from dipropylene glycol monobutyl ether (hereinafter referred to as “BDPG”) and tripropylene glycol monobutyl ether (hereinafter referred to as “BTPG”).

Oxypropylene group-containing glycol ether products that are available in the market differ in terms of the number of moles of PO adducted, purity, moisture content, acid value, and the like, depending on the supplier, but as mentioned above, some of these products, such as propylene glycol monoallyl ether, dipropylene glycol monoallyl ether, and tripropylene glycol monoallyl ether, can contain allyl group-containing impurities derived from their synthetic reactions.

These allyl group-containing impurities have allyl groups only at one end of the molecule. If these allyl group-containing impurities are present in the polymerization reaction system of a polyether-polysiloxane block copolymer that uses a dimethylpolysiloxane raw material having a silicon atom-bonded hydrogen atom at both ends of the molecular chain and a polyether raw material having alkenyl groups at both ends, the addition reaction of allyl group-containing impurities to the silicon atom-bonded hydrogen atoms at the ends of the dimethylpolysiloxane causes side reactions that stop the terminal reactions. This may inhibit the intended block copolymerization and significantly reduce the molecular weight and viscosity of the final copolymer.

Therefore, when the aforementioned oxypropylene group-containing glycol ether is used as a reaction solvent or diluent for a polyether-polysiloxane block copolymer, especially when used as a reaction solvent, the allyl group-containing impurities that cause side reactions must be reduced as much as possible, from the perspective of quality and production control of the resulting copolymers. However, these allyl group-containing impurities are extremely close in molecular weight and chemical structure to the main component oxypropylene group-containing glycol ether, and are difficult to efficiently remove by ordinary purification operations such as distillation. In addition, the propylation reaction described in Patent Document 5 is expensive and difficult to use in mass production from an industrial perspective.

The amount of these allyl group-containing impurities can be easily identified, for example, by nuclear magnetic resonance (NMR) analysis using 13C as the nuclear species. In particular, when more than 0.01 mol % of allyl group per mole of oxypropylene group is included, there is concern that side reactions that inhibit the formation of polyether-polysiloxane block copolymers may be induced when used as a reaction solvent or diluent for a polyether-polysiloxane block copolymer. Reference Data 1 and Reference Data 2 in the present application example show the results of determining the amount of allyl group containing-impurities as a ratio to the number of moles of allyl groups per mole of oxypropylene groups in commercial products of oxypropylene group-containing glycol ether. The commercial products contain 0.010 to 0.20 mol % of allyl groups per mole of oxypropylene groups, and in particular, 0.10-0.20 moles of allyl groups are included in low-priced BDPG or BDPG products. When used as a reaction solvent or diluent for polyether-polysiloxane block copolymers, it is highly desirable to reduce and purify the allyl group containing-impurities by the production method of the present invention as described below.

The manufacturing method of the present invention includes a reaction process for reducing allyl group containing-impurities by converting to hydrosilylation reaction products using a hydrosilylation reaction, a process for purifying oxypropylene group-containing glycol ether by separating the reaction products using a separation operation such as distillation after the reaction process, and a process for adding an antioxidant to the oxypropylene group-containing glycol ether at any timing.

### [Hydrosilylation Reaction Step]

The hydrosilylation reaction step of the present invention is a step in which an allyl group-containing impurity in an oxypropylene group-containing glycol ether is reacted with a silicon atom-bonded hydrogen atom (Si—H)-containing compound by hydrosilylation reaction, and an object thereof is to inactivate the allyl group in the allyl group-containing impurity by the reaction, and to convert the reaction product and oxypropylene group-containing glycol ether into components with significantly different molecular weights by adding an Si—H-containing compound, thereby making the impurities easy to purify by a known separation operation such as distillation. The addition of Si—H-containing compounds to allyl group-containing impurities does not cause side reactions in polyether-polysiloxane block copolymers, so if commercially and quality-wise acceptable, the reaction products can be used without separation, and the oxypropylene group-containing glycol ether may be used.

Silicon atom-bonded hydrogen atom (Si—H)-containing compounds can be used so long as there is at least one or more Si—H group in the molecule, and examples of commonly known Si—H-containing organic silicon compounds include: hydride silanes; hydride siloxane oligomers; cyclic, chain, and resinous hydrogen polysiloxanes; hydrogen silsesquioxane, and the like. However, when separating the hydrosilylation reaction products with allyl group-containing impurities by distillation, if the SiH-containing organosilicon compounds are non-volatile, there is an advantage that the distillation operation with unreacted Si—H-containing compounds will be extremely easy. Therefore, the Si—H-containing compound used in the production method of the present invention should be non-volatile, and industrially, the use of methylhydrogen polysiloxane with a viscosity or degree of polymerization that causes non-volatility can be suggested.

The amount of Si—H-containing compound used is an amount that provides 1 mole or more of Si—H for 1 mole of allyl groups in the allyl group containing-impurity in the oxypropylene group-containing glycol ether, and the amount of Si—H used can be in a range of 1 to 20 moles, 2 to 15 moles, or 3 to 10 moles, and a small excess to excess of Si—H can be used. As described above, unreacted Si—H-containing compounds such as methylhydrogenpolysiloxane can be separated together with the hydrosilylation reaction products from the oxypropylene group-containing glycol ether by distillation or the like.

The hydrosilylation reaction process proceeds in the presence of an effective amount of hydrosilylation reaction catalyst. The hydrosilylation reaction catalyst is not restricted as long as uniform dispersion in an oxypropylene group-containing glycol ether is possible, and the hydrosilylation reaction catalyst can be selected from among known hydrosilylation reaction catalysts for use in the present invention. Specific examples of the hydrosilylation reaction catalyst can include fine particulate platinum adsorbed on silica fine powder or a carbon powder carrier, chloroplatinic acids, alcohol-modified chloroplatinic acids, olefin complexes of chloroplatinic acid, coordinate compounds of chloroplatinic acid and vinyl siloxane, platinum such as platinum black, and the like.

In the manufacturing method of the present invention, a particularly preferable hydrosilylation reaction catalyst is a neutral platinum complex catalyst, and 1,3-divinyl-1,1,3,3-tetramethyldisiloxane platinum complex is particularly preferable. On the other hand, when an acidic hydrosilylation reaction catalyst such as chloroplatinic acid or alcohol-modified chloroplatinic acid is used, it may be necessary to use or increase the amount of the buffering agent component such as a potassium salt or sodium salt.

However, if the total mass of the oxypropylene group-containing glycol ether is assumed to be 100 mass %, the amount of metallic atoms (in particular, platinum group metallic atoms) in the hydrosilation catalysts is within a range of 0.1 to 1000 ppm by mass, preferably 1 to 100 ppm by mass. Note that the hydrosilylation reaction catalyst is basically nonvolatile, so it can be separated from the oxypropylene group-containing glycol ether by distillation, filtration, or other known separation method after the reaction.

The conditions for the hydrosilylation reaction in the present invention can be selected arbitrarily, but if necessary, a small amount (1 pp to 1 mass %) of an antioxidant such as BHT (2,6-di-t-butyl-p-cresol or dibutylhydroxytoluene) or tocopherol (vitamin E) or the like can be added to the oxypropylene group-containing glycol ether, followed by heating and stirring at room temperature to 200° C., suitably at 50 to 100° C. under an inert gas atmosphere such as nitrogen. Note that the antioxidant may be added after the hydrosilylation reaction is completed. The reaction time can be selected based on the reaction scale, amount of catalyst used, and reaction temperature, and is generally within a range of several minutes to several hours.

The reaction is carried out with a small excess of Si—H groups relative to C═C groups in the present invention, so the endpoint of the hydrosilylation reaction can be confirmed by the reduction of the Si—H bond absorption by infrared spectroscopy (IR) by a predetermined amount or by the following alkaline decomposition gas generation method, where hydrogen gas generation is reduced by a calculated amount compared to the initial value. The amount of hydrogen gas generated can also be determined by analyzing the Si—H-containing compounds, which are the reaction raw materials, using the same methods. The following is a summary thereof.

<Alkali decomposition gas generating method: Method of reacting at room temperature a 28.5 mass % caustic potash ethanol/water mixed solution with a solution where a sample is dissolved in toluene or IPA, collecting the generated hydrogen gas in a collection tube, and then measuring the volume thereof>

### [Purity]

In the production method of the present invention, the purity of the oxypropylene group-containing glycol ether after the aforementioned hydrosilylation reaction step is preferably 90 mass % or more, and the glycol ether is preferably substantially free of allyl group containing-impurities. Here, the term “substantially free of allyl group-containing impurities” is satisfied if the aforementioned Si—H-containing compound is added in a stoichiometrically equal amount (in other words, a molar ratio of 1:1) or more to the allyl groups in the allyl group-containing impurities, and, as mentioned above, the hydrosilylation reaction product is no longer classified as an allyl group-containing impurity. The purity of the oxypropylene group-containing glycol ether is preferably at least 95 mass % after the purification process described below, where the hydrosilylation reaction products, unreacted Si—H containing compounds, hydrosilylation reaction catalysts, and the like are separated. Purification to 95 to 99.9 mass % is especially preferable from the viewpoint of use as a reaction solvent or diluent for polyether-polysiloxane block copolymers.

### [Purification Process]

In the method for producing oxypropylene group-containing glycol ether of the present invention, it is preferable to separate the oxypropylene group-containing glycol ether from the hydrosilylation reaction products after the aforementioned hydrosilylation reaction process in order to purify the oxypropylene group-containing glycol ethers to a high purity that is practically free of allyl group containing-impurities (=allyl group-free). Note that in the purification process, unreacted Si—H-containing compounds and the hydrosilylation reaction catalyst are preferably removed. These components may also cause side reactions when used as reaction solvents or diluents for polyether-polysiloxane block copolymers.

The separation means used in the purification process can be selected according to the properties of the Si—H-containing compound used in the hydrosilylation reaction process described above (especially molecular weight and volatility), and the properties of the hydrosilylation reaction product (especially molecular weight and volatility). However, it is particularly preferable to include a distillation step because the reaction can be designed so that the molecular weight and boiling point of the hydrosilylation reaction products differ significantly from those of the oxypropylene group-containing glycol ether. In particular, if non-volatile components are selected as the Si—H-containing compounds, the hydrosilylation reaction products are often practically non-volatile, and thus there is an advantage that only the oxypropylene group-containing glycol ether can be separated and purified to extremely high purity, even with a simple distillation process and equipment.

The distillation step can be performed by a known method. If the Si—H-containing compounds and hydrosilylation reaction products are practically non-volatile, they can often be purified to a high purity simply by heat distillation under reduced pressure conditions. For industrial applications, the distillation pressure should be, for example, 0.01 mmHg or higher and 100 mmHg or lower, but 50 mmHg or lower is preferred, and 10 mmHg or lower is more preferred. Additionally, the distillation temperature can be, for example, 60 to 200° C., preferably 100 to 190° C., and more preferably 120 to 180° C. The reduced pressure distillation time is generally designed to be in a range of 30 minutes to several tens of hours, depending on the purification scale and reduced pressure conditions.

The purification process can be performed by distillation alone, but if coloration or solid residue of oxypropylene group-containing glycol ether is observed, filtration can be performed under normal temperature and pressure, or under pressurized conditions or under reduced pressure conditions using known filtration materials such as filter paper, zeta potential adsorption filters, bag filters, cartridge filters, adsorbents, filter aids, and the like.

### [Antioxidant Addition Step]

The method of producing oxypropylene group-containing glycol ether of the present invention may include a process of adding known antioxidants at any time. The addition of antioxidants may be before or after the hydrosilylation reaction. In addition, antioxidants may be added to the oxypropylene group-containing glycol ether that has been separated from the other components after the purification process. In addition, the antioxidant addition operation does not have to be performed only once, but can be performed multiple times, and is not restricted. As an example, the same or a different antioxidant may be added to the oxypropylene group-containing glycol ether after the purification process after adding the antioxidant to the reaction system before the hydrosilylation reaction. In addition to preventing oxidative degradation of the oxypropylene group-containing glycol ether, the addition of an antioxidant is highly desirable for preventing oxidative degradation of the copolymer during and after polymerization when the oxypropylene group-containing glycol ether is used as a reaction solvent or diluent for the polyether-polysiloxane block copolymers.

The type of antioxidant is not restricted as long as the antioxidant does not adversely affect the hydrosilylation reaction. Thus, antioxidants such as phenols and vitamins can be added to improve oxidation stability. For example, BHT, vitamin E, and the like can be used as the antioxidant.

The amount of antioxidant added is arbitrary, but is preferably in a range of 1 to 10000 ppm (=1.0 mass %) with respect to the oxypropylene group-containing glycol ether, and particularly preferably in a range of 50 to 500 ppm.

### [Buffer]

The oxypropylene group-containing glycol ether of the present invention may include a known buffering agent in the manufacturing process described above. By using the buffering agent, in particular, an increase in the overall acid value due to the oxypropylene group-containing compound, Si—H containing compound, the hydrosilylation reaction catalyst, and the like can be suppressed, and quality can be stabilized in some cases. Examples of buffers include known potassium salts such as potassium carbonate and known sodium salts such as sodium acetate. In addition to stabilizing the quality of the actual oxypropylene group-containing glycol ether, some soluble potassium salts function as buffers and may further improve the foam conditioning performance when the oxypropylene group-containing glycol ether is used as a reaction solvent or diluent of polyether-polysiloxane block copolymers.

A known curing inhibitor may be added to the kettle residue after the hydrosilylation reaction or after purification, or to the drum-off kettle residue, in order to terminate the hydrosilylation reaction and deactivate the catalyst. These curing inhibitors can be added in small amounts as described in Japanese Unexamined Patent Application 2007-308542, and examples include acetylene compounds, enyne compounds, organic nitrogen compounds, organic phosphorus compounds, oxime compounds, phosphorus compounds, 1,2-bis(diphenylphosphino)ethane, 1,3-bis(diphenylphosphino)propane, and the like.

### [Production Method of Polyether-Polysiloxane Block Copolymer Composition]

The oxypropylene group-containing glycol ether obtained by the above production method is suitable for use as a reaction solvent or diluent for polyether-polysiloxane block copolymers obtained by the hydrosilylation reaction of organopolysiloxanes containing SiH groups at both ends and polyethers containing methallyl groups at both ends. In particular, the oxypropylene group-containing glycol ethers obtained by the above production method are highly pure and virtually free of allyl group containing-impurities (=allyl group free) using a simple method, so only compounds with an allyl group at one end derived from such impurities are added to the organopolysiloxanes containing SiH groups at both ends. This has the practical benefit of enabling stable production of higher molecular weight and higher viscosity polyether-polysiloxane block copolymers because allyl groups are only added to the organopolysiloxane containing SiH groups at both ends, and side reactions that inhibit block copolymer formation are suppressed. Note that the above reaction can be performed without restriction, and the glycol ethers in the reaction proposed by the applicants in Patent Document 10 can be replaced using the production method of the present application. In particular, the average molecular weight of the copolymer can be designed by adjusting the molar ratio (reaction ratio) between the organopolysiloxane containing SiH groups at both ends and the polyether containing alkenyl groups at both ends. Furthermore, surface activating performance, affinity to a urethane foam system, and the like can be controlled based on the EO % or size of a polyether moiety, and introduction of a hydroxyl group or hydrophobic group to a copolymer terminal moiety. The copolymer can be used in various types of polyurethane foam formulations with excellent effects as surfactants for foam control or foam stabilization.

### [Polyurethane Foam-Forming Composition]

The oxypropylene group-containing glycol ether obtained by the above production method can be suitably used as a reaction solvent or diluent for (AB)n-type polyether-modified silicone foaming agents containing the polyether-polysiloxane block copolymers described in Patent Document 10, and the like. Therefore, the polyurethane foam forming composition can be produced by mixing at least (a) polyol, (b) polyisocyanate, (c) catalyst, (d) (AB)n polyether-modified silicone foaming agent containing an oxypropylene group-containing glycol ether obtained by the above manufacturing method, and optionally (e) at least one additive component selected from foaming agents other than component (d), blowing agents, diluents, chain elongating agents, crosslinking agents, water, non-aqueous blowing agents, fillers, reinforcing agents, pigments, dyes, colorants, flame retardants, antioxidants, anti-ozone agents, UV stabilizers, antistatic agents, fungicides, and antibacterial agents.

### [Other Applications]

The oxypropylene group-containing glycol ethers obtained by the above production method are highly pure and practically free of allyl group-containing impurities (=allyl group-free), and can be used for known applications of oxypropylene group-containing glycol ethers without any particular restriction.

The polyether-polysiloxane block copolymer obtained by the above production method has the advantage that side reactions are suppressed and stably producing a high molecular weight and high viscosity polyether-polysiloxane block copolymer is easy. The method can be used for surfactants, foaming agents, fiber lubricating or softening agents, surface treatment or coating agents, and reactive raw materials for other polymeric materials, without any particular limitation.

In particular, the polyether-polysiloxane block copolymer compositions containing oxypropylene group-containing glycol ethers as a reaction solvent or diluent obtained by the manufacturing method of the present invention are useful as surfactants for industrial use, and target products include paints, coating agents, building materials, hydrophilic agents, surface treatment agents, and foaming resin compositions, without limitations in particular. Furthermore, due to the function as a surfactant, the composition is particularly useful as an additive for paints, emulsifiers, solubilizers, foaming agents for polyurethane foams, and additives to premix solutions for polyurethane foams.

### EXAMPLES

Hereinafter, the present invention will be further described in detail based on Examples and Comparative Examples, but the present invention is not limited thereto. Note that in the following composition formulas, a Me₃SiO group (or Me₃Si group) is expressed simply as “M”, a Me₂SiO group is expressed as “D”, a MeHSiO group is expressed as “M^{H}”, and units where a methyl group in the M or D is modified by any substitution group are expressed as M^{R} and D^{R}. IPA represents isopropanol, MeOH represents methanol, BDPG represents dipropylene glycol monobutyl ether, BTPG represents tripropylene glycol monobutyl ether, EO represents ethylene oxide or an oxyethylene group, and PO represents propylene oxide or an oxypropylene group. In addition, “%” in the test examples refers to mass %, unless otherwise specified. The number of moles of allyl groups per mole of oxypropylene groups (PO) (mol %) shown in the reference data in Table 1 and Table 2 are the values determined based on an analysis of commercial products by the inventors using 13C-NMR.

The following reference data 1 (Table 1) and reference data 2 (Table 2) show the differences in quality of BDPG by supplier and the differences in quality of BTPG by supplier. Company H products are high-priced products and Company L products are low-priced products. In the following test examples, when BDPG made by Company H or Company L is used, it is denoted as “BDPG (Company H)” or “BDPG (Company L)” and when BTPG made by Company H or Company L above is used, it is denoted as “BTPG (Company H)” or “BTPG (Company L)”. While it can be determined from the CoA description that these products differ in purity, moisture content, acid number, and the like, there was no description of the amount of allyl group-containing impurities, so the amount of allyl groups/PO (mol %) identified by 13C-NMR are listed in the tables.

### <Reference Data 1>

TABLE 1
Difference in quality according to supplier of BDPG

| | Company H | Company L | |
|---|---|---|---|
| Items | (high price) | (low price) | Data Source |
| Purity [wt. %] | 99.7 | 99.1 | Table of Analysis |
| Moisture [wt. %] | 0.02 | 0.01 | (CoA) provided |
| Hue (APHA) | 10 | 3 | by each supplier |
| Acid value [wt. %] | 0.0010 | <0.01 | |
| Specific gravity | 0.914 | 0.90-0.92 | |
| (25/25° C.) | | | |
| Allyl group/PO | 0.015 | 0.20 | By the inventors |
| [mol %] | | | ¹³C NMR analysis |

### <Reference Data 2>

TABLE 2
Difference in quality according to supplier of BTPG

| | Company H | Company L | |
|---|---|---|---|
| Items | (high price) | (low price) | Data Source |
| Purity [wt. %] | 99.8 | 97.0 | Table of Analysis |
| Moisture [wt. %] | 0.02 | 0.02 | (CoA) provided |
| Hue (APHA) | 10 | 7 | by each supplier |
| Acid value [wt. %] | 0.0010 | <0.01 | |
| Specific gravity | 0.934 | 0.925-0.945 | |
| (25/25° C.) | | | |
| Allyl group/PO | 0.01 | 0.10 | By the inventors |
| [mol %] | | | ¹³C NMR analysis |

In the following Examples 1 to 3, the allyl group-containing impurities in BTPG (Company L) and BDPG (Company L) were reduced and purified by the production method described in the present invention.

In addition, in Examples 4 to 6, BTPG or BDPG obtained by the manufacturing process of the present invention was utilized as a reaction solvent or diluent to synthesize (AB)n-type polyether-modified silicone foaming agents, which are polyether-polysiloxane block copolymer compositions.

As a reference, commercially available BTPG or BDPG was used as a reaction solvent or diluent in Reference Examples 1 to 3 to synthesize (AB)n-type polyether-modified silicone foaming agents.

The raw material components used in these tests were as follows.
(a1-1) bismethallylpolyether expressed by the average compositional formula CH₂═C(CH₃)CH₂—O(C₂H₄O)₃₉(C₃H₆O)₂₀—CH₂—C(CH₃)═CH₂ (degree of unsaturation 0.68 meq/g, hydroxyl value 0.8 mg-KOH/g)
(a1-2) bismethallylpolyether expressed by the average compositional formula CH₂═C(CH₃)CH₂—O(C₂H₄O)₃₉(C₃H₆O)₂₀—CH₂—C(CH₃)═CH₂

(degree of unsaturation 0.66 meq/g, hydroxyl value 1.1 mg-KOH/g)

* Remarks: (a1-1) to (a1-2) correspond to raw materials of bismethallyl polyethers of different lots. Herein, a polyether portion is a random adduct of ethylene oxide and propylene oxide.
(a2-1) Methylhydrogen polysiloxane (silicon-bonded hydrogen content 0.125%) with average composition M^{H}D₂₀M^{H}, which has been treated in advance to remove low boiling point substances
(a2-2) Methylhydrogen polysiloxane (silicon-bonded hydrogen content 0.128%) with average composition M^{H}D₂₀M^{H}, which has been treated in advance to remove low boiling point substances
(a2-3) Methylhydrogen polysiloxane (silicon-bonded hydrogen content 0.129%) with average composition M^{H}D₂₀M^{H}, which has been treated in advance to remove low boiling point substances

*Remarks: (a2-1) to (a2-3) correspond to raw materials from different lots of methylhydrogenpolysiloxane.

Pt catalyst (hydrosilylation reaction catalyst): 1,3-divinyl-1,1,3,3-tetramethyldisiloxane platinum complex was used (PT concentration 4.3 wt %)

### Example 1: Production of BTPG (Example 1) by Purification of BTPG (Company L)

In a 2 L reactor, 18.0 g (1.0% of the total) of (a2-3) methylhydrogenpolysiloxane, 1795.6 g (99.0% of the total) of BTPG (Company L), and 0.43 g of hydrosilylation reaction catalyst were added and heating was started while stirring under a nitrogen gas flow. The calculated SiH/C═C molar ratio at this time was 6.3. Aging at 80 to 90° C. for 3 hours was performed to consume allyl groups derived from the allyl group containing-impurities in the BTPG. The calculated BTPG purity at this stage was 96%. Here, this purity is synonymous with the purity in the CoA of the supplier.

The entire area from the upper connecting tube of the separable flask to the distillation head was then wrapped with a ribbon heater and covered with thermal insulation to prevent air cooling due to outside temperature. BTPG was collected in a flask by setting the ribbon heater at 130° C. and the oil bath at 150 to 170° C. and reducing the pressure in the flask to less than 10 mmHg while stirring under nitrogen flow. Accumulation began at an internal liquid temperature of around 150° C. By keeping this condition for a total of about 12 hours, 1589 g of BTPG (Example 1) was obtained.

### Example 2: Production of BTPG (Example 2) by Purification of BTPG (Company L)

In a 3 L reactor, 28.0 g (1.0% of the total) of (a2-3) methylhydrogenpolysiloxane and 2804.6 g (99.0% of the total) of BTPG (Company L) were added and stirring was performed under a nitrogen gas flow at a temperature of 75° C. The calculated SiH/C═C molar ratio at this time was 6.3. 0.67 g of hydrosilylation reaction catalyst was added and aged at 75 to 90° C. for 5 hours to consume the allyl groups derived from the allyl group-containing impurities in the BTPG. The calculated BTPG purity at this stage was 96%. Here, this purity is synonymous with the purity in the CoA of the supplier.

The entire area from the upper connecting tube of the separable flask to the distillation head was then wrapped with a ribbon heater and covered with thermal insulation to prevent air cooling due to outside temperature. BTPG was collected in a flask by setting the ribbon heater at 130° C. and the oil bath at 150 to 170° C. and reducing the pressure in the flask to less than 10 mmHg while stirring under nitrogen flow. Accumulation began at an internal liquid temperature of around 150° C. By keeping this condition for a total of about 16 hours, 2695 g of BTPG (Example 2) was obtained.

### Example 3: Production of BDPG (Example 3) by Purification of BDPG (Company L)

In a 2 L reactor, 12.0 g (1.0% of the total) of (a2-3) methylhydrogenpolysiloxane, 1188.0 g (99.0% of the total) of BDPG (Company L), and 0.28 g of hydrosilylation reaction catalyst were added and heating to 75° C. was performed while stirring under a nitrogen gas flow. The calculated SiH/C═C molar ratio at this time was 6.4. Aging at 75 to 90° C. for 2.5 hours was performed to consume allyl groups derived from the allyl group-containing impurities in the BDPG. The calculated BDPG purity at this stage was 98%. Here, this purity is synonymous with the purity in the CoA of the supplier.

The entire area from the upper connecting tube of the separable flask to the distillation head was then wrapped with a ribbon heater and covered with thermal insulation to prevent air cooling due to outside temperature. BDPG was collected in a flask by setting the ribbon heater at 125° C. and the oil bath at 125 to 135° C. and reducing the pressure in the flask to less than 12 mmHg while stirring under nitrogen flow. Accumulation began at an internal liquid temperature of around 125° C. By keeping this condition for about 2.5 hours, 1167 g of BDPG (Example 3) of the present invention was obtained.

The following is a summary of the results of the determination of residual allyl groups in the high-purity BTPG of Examples 1 and 2 obtained by the production method of the present invention, in comparison with BTPG (Company H) and BTPG (Company L). In these samples, there was no sign of propenyl groups produced by isomerization of allyl groups.

TABLE 3
Analysis results of allyl groups in BTPG (¹³CNMR)

| | | Company L | | |
|---|---|---|---|---|
| | | (low price) | Example 1 | Example 2 |
| | | Before | After | After |
| | | purification | purification | purification |
| | Company H | of the | of the | of the |
| Items | (High price) | invention | invention | invention |
| Allyl | 0.01 | 0.10 | 0.003 | 0.01 |
| group/PO | | | | |
| [mol %] | | | | |

The following is a summary of the results of the determination of residual allyl groups in the high-purity BDPG of Example 3 obtained by the production method of the present invention, in comparison with BDPG (Company H) and BDPG (Company L). In these samples, there was no sign of propenyl groups produced by isomerization of allyl groups.

TABLE 4
Analysis results of allyl groups in BDPG (13CNMR)

| | | Company L (low price) | Example 3 |
|---|---|---|---|
| | Company H | Before purification of | After purification |
| Items | (High price) | the invention | of the invention |
| Allyl | 0.015 | 0.20 | 0.03 |
| group/PO | | | |
| [mol %] | | | |

Next, BTPG (Company H), BTPG (Company L), and BTPG (Example 1) were used as reaction solvents and diluents to synthesize (AB)n polyether modified silicone foaming agents. Here, the molar ratio of C═C and Si—H groups in the polyether raw material was fixed at C═C/SiH=1.05 for each test.

### Reference Example 1

A 500 mL reaction vessel was charged with 34.18 g of (a1-1) bismethallylpolyether (containing 500 ppm of natural vitamin E), 0.04 g of 10 wt. % sodium acetate in MeOH, and 0.10 g of natural vitamin E, and heating was started while stirring under nitrogen gas flow. MeOH was removed out of the system by a stripping operation for 1 hour under the conditions of 30 to 60° C. and 58 mmHg. The pressure was restored, 17.17 g of (a2-3) methylhydrogenpolysiloxane and 154.05 g of BTPG (company H) were added under stirring, 0.18 mL of hydrosilation catalyst was also added, and the reaction was carried out at 60 to 80° C. for 2 hours, and as a result, the reaction was substantially complete. Thereby, a transparent liquid (AB)n type polyether modified silicone foaming agent “970 mm2/s, (25° C.)” including the straight chain organopolysiloxane-polyether block copolymer at least containing a structural unit expressed by the average composition formula:

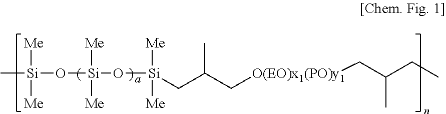

(where a=20, x1=39, y1=20, and n>10) and

BTPG was obtained at a 25:75 ratio.

### Reference Example 2

A 1 L reaction vessel was charged with 58.80 g of (a2-2) methylhydrogenpolysiloxane, 116.20 g of (a1-1) bismethallylpolyether (containing 500 ppm of natural vitamin E), and 0.14 g of 10 wt. % sodium acetate in MeOH, and heating was started while stirring under nitrogen gas flow. MeOH was removed out of the system by a stripping operation for 30 minutes under the conditions of 70 to 80° C. and 20 to 25 mmHg. The pressure was restored, 525 g of BTPG (company L) was added while stirring, 0.16 g of hydrosilation catalyst was also added, and the reaction was carried out at 80 to 90° C. for 3 hours, and as a result, the reaction was substantially complete. Thereby, a transparent liquid (AB)n type polyether modified silicone foaming agent “171 mm²/s, (25° C.)” including the straight chain organopolysiloxane-polyether block copolymer at least containing a structural unit expressed by the average composition formula:

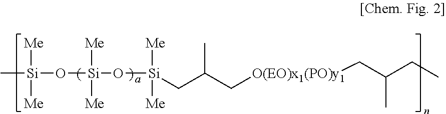

(where a=20, x1=39, y1=20, and n≈8) and

BTPG was obtained at a 25:75 ratio.

### Example 4

A 1 L reaction vessel was charged with 66.96 g of (a2-1) methylhydrogenpolysiloxane, 133.04 g of (a1-2) bismethallylpolyether (containing 500 ppm of natural vitamin E), and 0.16 g of 10 wt. % sodium acetate in MeOH, and heating was started while stirring under nitrogen gas flow. MeOH was removed out of the system by a stripping operation for 40 minutes under the conditions of 50 to 75° C. and 5 mmHg. The pressure was restored, 600 g of BTPG (Example 1) was added while stirring, 0.048 g of hydrosilation catalyst was also added, and the reaction was carried out at 70 to 80° C. for 3 hours, and as a result, the reaction was substantially complete. Thereby, a transparent liquid (AB)n type polyether modified silicone foaming agent “626 mm²/s, (25° C.)” including the straight chain organopolysiloxane-polyether block copolymer at least containing a structural unit expressed by the average composition formula:

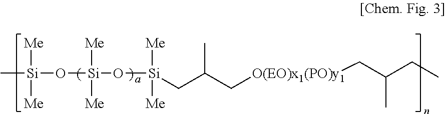

(where a=20, x1=39, y1=20, and n>10) and

BTPG was obtained at a 25:75 ratio.

Next, BDPG (Company H) and BDPG (Example 3) were used as reaction solvents and diluents to synthesize (AB)n polyether modified silicone foaming agents.

### Reference Example 3

A 1 L reaction vessel was charged with 66.96 g of (a2-1) methylhydrogenpolysiloxane, 133.04 g of (a1-2) bismethallylpolyether (containing 500 ppm of natural vitamin E), 0.16 g of 10 wt. % sodium acetate in MeOH, and 200 g of BDPG (Company H), and heating was started while stirring under nitrogen gas flow. MeOH was removed out of the system by a stripping operation for 40 minutes under the conditions of 70 to 80° C. and 16 mmHg. The pressure was restored, and then 0.106 mL of the hydrosilation catalyst was added, and the reaction was carried out at 70 to 80° C. for 3 hours. As a result, the reaction was completed. 400 g of BDPG (Company H) was further added and mixed to obtain a clear liquid (AB)n type polyether-modified silicone foaming agent containing linear organopolysiloxane-polyether block copolymer and BDPG in a 25:75 ratio, similar to Reference Example 3 “427 mm²/s (25° C.)”. Here, the molar ratio of C═C and Si—H groups in the polyether raw material was C═C/SiH=1.05.

### Example 5

A 500 mL reaction vessel was charged with 25.56 g of (a2-1) methylhydrogenpolysiloxane, 49.44 g of (a1-2) bismethallylpolyether (containing 500 ppm of natural vitamin E), 0.06 g of 10 wt. % sodium acetate in MeOH, and 75.0 g of BDPG (Example 3), and heating was started while stirring under nitrogen gas flow. MeOH was removed out of the system by a stripping operation for 15 minutes under the conditions of 50 to 75° C. and 15 mmHg. The pressure was restored, and then 0.04 mL of the hydrosilation catalyst was added, and the reaction was carried out at 70 to 80° C. for 3 hours. As a result, the reaction was completed. 150 g of BDPG (Example 3) was further added and mixed to obtain a clear liquid (AB)n type polyether-modified silicone foaming agent containing linear organopolysiloxane-polyether block copolymer and BDPG in a 25:75 ratio, similar to Reference Example 3 “650 mm²/s (25° C.)”. Here, the molar ratio of C═C and Si—H groups in the polyether raw material was C═C/SiH=1.02.

### Summary

Examples 1 to 3 show that it was possible to purify low-cost BDPG or BTPG using the production method of the present invention, and to obtain an oxypropylene group-containing glycol ether with sufficiently low allyl group/PO (mol %) and sufficiently reduced allyl group containing-impurities for practical use, which are comparable to high-priced and high-purity commercial products.

Furthermore, as shown in Examples 4 and 5, synthesis of an (AB)n-type polyether-modified silicone foaming agent was performed using BDPG or BTPG obtained using the above production method as a reaction solvent and diluent. The foaming agents in these examples have sufficient viscosity and suppressed side reactions as compared to the synthesis examples using a high-priced and high-purity commercial product from Company H, which were conducted as reference tests (Reference Examples 1 and 3), and the production method of the present invention enabled stable production of (AB)n-type polyether modified silicone foaming agents with excellent quality. On the other hand, when the low-priced BTPG made by Company L was used as a reaction solvent or diluent (Reference Example 2), the resulting (AB)n-type polyether-modified silicone foaming agent had significantly lower viscosity, and did not have sufficient foaming performance due to side reactions caused by allyl group containing-impurities. There is strong concern that production with stable quality would be difficult.

1. A method for producing an oxypropylene group-containing glycol ether where a hydrogen is substituted with a hydrocarbon group having 1 to 8 carbon atoms at one end and that has an alcoholic hydroxyl group at another end and where the number of repeating units of oxyalkylene with 2 to 4 carbon atoms is in a range of 1 to 3, the method comprising:
hydrosilylation reacting a silicon atom-bonded hydrogen atom (Si—H)-containing compound and an allyl group-containing impurity including an oxypropylene group-containing glycol ether that does not contain a heteroatom other than oxygen.

2. The method for producing an oxypropylene group-containing glycol ether according to claim 1, wherein the purity after the aforementioned hydrosilylation reaction process is 90 mass % or more and the glycol ether is substantially free of allyl group containing-impurities.

3. The method for producing an oxypropylene group-containing glycol ether according to claim 1, wherein a purification step for separating the oxypropylene group-containing glycol ether and the hydrosilylation reaction product is also performed after the aforementioned hydrosilylation reaction process.

4. The method for producing an oxypropylene group-containing glycol ether according to claim 3, wherein the purification step includes a distillation process of the glycol ether containing an oxypropylene group.

5. The method for producing an oxypropylene group-containing glycol ether according to claim 1, wherein a nonvolatile polysiloxane containing one or more Si—H groups per molecule is used as a silicon atom-bonded hydrogen atom (Si—H)-containing compound in the aforementioned hydrosilylation reaction step.

6. The method for producing an oxypropylene group-containing glycol ether according to claim 1, wherein the molar ratio of residual C═C groups to oxypropylene groups is 1/2500 (0.04 mol %) or less.

7. The method for producing an oxypropylene group-containing glycol ether according to claim 1, wherein the purity of the glycol ether containing an oxypropylene group obtained after the aforementioned hydrosilylation reaction step is 95 mass % or more.

8. The method of manufacturing an oxypropylene group-containing glycol ether according to claim 1, wherein the oxypropylene group-containing glycol ether is one or more types of glycol ether selected from propylene glycol monobutyl ethers, dipropylene glycol monobutyl ethers, tripropylene glycol monobutyl ethers, propylene glycol monomethyl ethers, dipropylene glycol monomethyl ethers, tripropylene glycol monomethyl ethers, propylene glycol monopropyl ethers, dipropylene glycol monopropyl ethers, tripropylene glycol monopropyl ethers, propylene glycol monoethyl ethers, dipropylene glycol monoethyl ethers, and tripropylene glycol monoethyl ethers.

9. The method of manufacturing an oxypropylene group-containing glycol ether according to claim 1, further comprising a step of adding 1 ppm to 1 mass % of an antioxidant to the glycol ether containing an oxypropylene group at optional timing.

10. A method for producing a polyether-polysiloxane block copolymer composition, wherein the oxypropylene group-containing glycol ether obtained by the method for producing an oxypropylene group-containing glycol ether according to claim 1 is used as a reaction solvent or diluent.

11. A method for producing a polyurethane foam folding composition, wherein the glycol ether containing an oxypropylene group obtained by the method for producing an oxypropylene group-containing glycol ether according to claim 1 used as a reaction solvent or diluent for a polyether-polysiloxane block copolymer.

12. One or more oxypropylene group-containing glycol ether or mixture thereof, wherein the molar ratio of residual C═C groups to oxypropylene groups is 1/2500 (0.04 mol %) or less, allyl group-containing impurities are not included, optionally including 1 ppm to 1% of an antioxidant, and a purity of 95 mass % or more, the oxypropylene group-containing glycol ethers selected from propylene glycol monobutyl ethers, dipropylene glycol monobutyl ethers, tripropylene glycol monobutyl ethers, propylene glycol monomethyl ethers, dipropylene glycol monomethyl ethers, tripropylene glycol monomethyl ethers, propylene glycol monopropyl ethers, dipropylene glycol monopropyl ethers, tripropylene glycol monopropyl ethers, propylene glycol monoethyl ethers, dipropylene glycol monoethyl ethers, and tripropylene glycol monoethyl ethers.
